(19) United States
(12) Patent Application Publication
(10) Pub. No.: US 2008/0275481 A1
(43) Pub. Date: Nov. 6, 2008

(54) Ultrasound guided percutaneous cutting tool with gradations and adjustable stop ring

(76) Inventor: Michael A. Scarpone, Bloomingdale, OH (US)

Correspondence Address: Donald W. Meeker;Patent Agent, 924 East Ocean Front #E, Newport Beach, CA, 92661, US

(21) Appl. No.: 11/800,160

(22) Filed: May 4, 2007

(51) Int. Cl. A61B 17/32; A61B 17/34

(52) U.S. Cl. 606/172; 604/264; 606/170

(57) ABSTRACT

A surgical cutting instrument which has an ultrasonic reflective coating and gradations thereon is used in association with an adjustable stop ring on the surgical cutting instrument for internal surgical procedures guided by ultrasonic scanning. The surgical cutting instrument is inserted into the introducer and the stop ring stops the surgical cutting instrument at a desired protruding distance of the cutting instrument into the area of the surgical procedure.

### CROSS-REFERENCE TO RELATED APPLICATIONS

Not Applicable.

### STATEMENT REGARDING FEDERALLY SPONSORED RESEARCH OR DEVELOPMENT

Not Applicable.

### THE NAMES OF THE PARTIES TO A JOINT RESEARCH OR DEVELOPMENT

Not Applicable.

### BACKGROUND OF THE INVENTION

1. Field of the Invention

The present invention relates to surgical instruments and particularly to a device and method for ultrasound guided percutaneous debridement of tendons and subsequent tendon release and ultrasound guided percutaneous release of plantar fascia; said device comprising a surgical cutting instrument which has a reflective coating and gradations thereon which are used in association with an adjustable stop ring on the surgical cutting instrument, the surgical cutting instrument being inserted into an introducer and the stop ring preventing the cutting instrument from traveling too far within an introducer with each pass of the surgical cutting instrument.

Description of Related Art Including Information Disclosed Under 37 CFR 1.97 and 1.98

While there are other devices and methods for performing tendon release and plantar fascia release, none provide the precision of the present device and method.

U.S. Pat. No. 6,228,049, issued May 8, 2001 to Schroeder, claims an introducer guide for introducing medical items into an internal tissue of a patient. The guide includes a cannula defining a lumen sized to receive the item and a lateral opening. A ramp is disposed within the lumen adjacent and inclined toward the lateral opening. A hub is attached to the second end of the cannula. The hub includes a gripping portion configured to be held when inserting and positioning the introducer. The gripping portion has a length sufficient for gripping and a tactile indicator disposed along substantially the entire length of the gripping portion on the hub. The indicator has a fixed angular relationship with the lateral opening.

U.S. Patent Application #20060190021, published Aug. 24, 2006 by Hausman, claims a cutting device for making subcutaneous incisions which is used in the release of tendons. The cutting device has a housing forming a handle. A tubular blade sub-assembly extends from the housing. A trocar passes through the tubular blade sub-assembly and is configured to be positioned in a first active position, with a tip of the trocar extending beyond the end of the tubular blade sub-assembly and a second retracted position where the tip of the trocar is held within the end of tubular blade sub-assembly. An optional depth stop indicator is also provided.

U.S. Patent Application #20030158521, published Aug. 21, 2003 by Ameri, discloses a trocar placement guide needle which reduces the need for trocar reinsertion attempts, by first ascertaining with the guide needle where a trocar should optimally be placed. In one embodiment, the guide needle is comprised of an elongate member including a proximal end and a sharp beveled distal tip for puncturing and cutting tissue, wherein the member includes an axial bore throughout the length of the member, a handle at the proximal end of the member, a means for indicating the depth to which the member has been inserted into the tissue, and a means for preventing unintended puncturing and cutting by the sharp beveled distal tip. Various embodiments are disclosed with different dimensions, means for precluding unintended puncturing or cutting, and depth indication means.

Two U.S. Patent Applications, #20030171743 published Sep. 11, 2003 and #20060189971 published Aug. 24, 2006 by Tasto, describe systems and methods for electrosurgical treatment of fasciitis by promoting blood flow to a target tissue. In one aspect, the invention involves canalizing or boring channels, divots, trenches or holes through an avascular connective tissue, or through a tissue having sparse vascularity, such as a tendon or a meniscus, in order to increase blood flow within the tissue. In one method, an active electrode is positioned in close proximity to a target site on a tendon, and a high frequency voltage difference is applied between the active electrode and a return electrode to selectively ablate tendon tissue at the target site, thereby forming a channel or void in the tendon. The active electrode(s) may be moved relative to the tendon during, or after, the application of electrical energy to damage or sculpt a void within the tendon, such as a hole, channel, crater, or the like. In another aspect of the invention, an electrosurgical probe is used to elicit a wound healing response in a target tissue, such as an injured tendon, in order to stimulate vascularization of the target tissue. The present invention may also be used for vascularization of a torn or damaged tissue in conjunction with a surgical repair procedure. An ultrasound tissue thickness measuring system may be incorporated within an electrosurgical instrument of the invention, e.g., probe or catheter, and thereby allow the surgeon to pre-set the depth of each channel using adjustable stop on handpiece.

Two U.S. Pat. No. 6,763,836 issued Jul. 20, 2004 and U.S. Pat. No. 6,805,130 issued Oct. 19, 2004 both to Tasto, are for methods for electrosurgical tendon vascularization and promoting blood flow to a target tissue. In one variation, the invention involves creating a pattern of voids in connective tissue, or through a tissue having sparse vascularity, such as a tendon or a meniscus, in order to increase blood flow within the tissue. This also includes using a template device to assist in the creation of the pattern of voids. Also included is an electrosurgical device with a self-contained fluid supply for providing conductive fluid to the target tissue or to active electrodes of the device. An ultrasound tissue thickness measuring system may be incorporated within an electrosurgical instrument of the invention, e.g., probe or catheter, and thereby allow the surgeon to pre-set the depth of each channel using adjustable stop on handpiece.

U.S. Pat. No. 6,602,248, issued Aug. 5, 2003 to Sharps, shows an apparatus and methods for treating an intervertebral disc by ablation of disc tissue. A method of the invention includes positioning at least one active electrode within the intervertebral disc, and applying at least a first high frequency voltage between the active electrode(s) and one or more return electrode(s), wherein the volume of the nucleus pulposus is decreased, pressure exerted by the nucleus pulposus on the annulus fibrosus is reduced, and discogenic pain of a patient is alleviated. In other embodiments, a curved or steerable probe is guided to a specific target site within a disc to be treated, and the disc tissue at the target site is ablated by application of at least a first high frequency voltage between the active electrode(s) and one or more return electrode(s). A method of making an electrosurgical probe is also disclosed. The shaft proximal end portion of electrosurgical probe includes a plurality of depth markings (shown as 903a-f in FIG. 32A). In other embodiments, other numbers and arrangements of depth markings may be included on shaft. For example, in certain embodiments, depth markings may be present along the entire length of shield, or a single depth marking may be present at shaft proximal end portion. Depth markings serve to indicate to the surgeon the depth of penetration of shaft into a patient's tissue, organ, or body, during a surgical procedure. Depth markings may be formed directly in or on shield, and may comprise the same material as shield. Alternatively, depth markings may be formed from a material other than that of shield. For example, depth markings may be formed from materials which have a different color and/or a different level of radiopacity, as compared with material of shield. For example, depth markings may comprise a metal, such as tungsten, gold, or platinum oxide (black), having a level of radiopacity different from that of shield. Such depth markings may be visualized by the surgeon during a procedure performed under fluoroscopy. In one embodiment, the length of introducer needle and shaft are selected to limit the range of shaft distal end beyond the distal tip of introducer needle. FIG. 32B shows a probe, wherein the shaft includes a mechanical stop

Three U.S. patents, U.S. Pat. No. 5,370,625 issued Dec. 6, 1994; U.S. Pat. No. 5,336,206 issued Aug. 9, 1994; and U.S. Pat. No. 5,217,441 issued Jun. 8, 1993 all to Shichman, describe a trocar guide tube positioning device. The trocar penetration depth indicator has a first, inner housing including a first threaded section and longitudinally extending gripping fingers, and a second, outer housing including a camming surface and a second threaded section to engage the first threaded section. The first and second housings slidably mount onto a trocar tube and can be secured relative to the guide tube by rotatably tightening the housings to cause the gripping fingers to firmly grip the trocar tube. Depth penetration indicia is provided on the guide tube surface and sufficient guide tube grasping force is obtained to prevent the depth indicator from being dislodged during trocar insertion. A guide tube positioning device is provided to engage the penetration depth indicator and to secure the guide tube relative to the surrounding skin of the patient in order to prevent inadvertent withdrawal or further insertion of the trocar.

U.S. Pat. No. 6,270,484, issued Aug. 7, 2001 to Yoon, discloses a safety penetrating instrument for penetrating an anatomical wall or a body lumen comprises an elongated penetrating member having proximal and distal ends. The distal end of the elongated penetrating member has a sharp or blunt tip for piercing the anatomical wall or penetrating the body lumen. A covering for the tip comprises an expandible sponge material having a first state with a first hardness and a second state with a second hardness less than the first hardness. Hydrating the covering material causes it to transition from the first hardness state to the second hardness state so that when the distal end of the elongated penetrating member passes from a position outside the anatomical wall or body lumen to a position inside the anatomical wall or lumen, the covering transitions from the first state to the second state to form a relatively soft protective member for the sharp or blunt tip. A method of safety penetration comprises the steps of covering the tip with an expandible sponge material and hydrating the covering during penetration of the anatomical wall or body lumen. The safety penetrating instrument may comprise a trocar with a portal sleeve, a cannula, needle, catheter or the like and may be provided with devices for optical viewing, imaging and/or sensing parameters, such as pressure, temperature, pH and other chemistry. A scale may be inscribed or otherwise affixed to the exterior peripheral surface of the sleeve adjacent the distal end thereof to facilitate determination of the depth of penetration of the instrument.

U.S. Pat. No. 3,993,079, issued Nov. 23, 1976 to de Gatztanondo, indicates a device for percutaneous paracentesis, injection, drainage and catheterization comprising a hypodermic needle having means for attachment to a syringe, with a central body portion having a reference mark and a smaller diameter leading end portion extending therefrom, its end terminating in a cutting bevel and a trocar adapted to surround the central body portion of the needle and capable of sliding thereon using it as a guide, having a graduated scale related to the overall length of the needle and having its end also terminating in a cutting bevel.

U.S. Pat. No. 5,279,567, issued Jan. 18, 1994 to Ciaglia, puts forth a trocar and tube with pressure signal. The trocar is slidably received within a thoracotomy tube. The thoracotomy tube is formed of plastic and is relatively flexible. The thoracotomy tube is provided with openings around the periphery of the tube at the distal end thereof. The thoracotomy tube also is provided with screw threads which extend from the distal end around the end portion of the tube extending approximately centimeter from the distal end of the thoracotomy tube. The thoracotomy tube further has markings along the length thereof to indicate the penetration of the end of the thoracotomy tube within the pleural cavity. A radiopaque stripe (not shown) extending along the length of the thoracotomy tube also provides an indication of the extent of penetration of the distal end of the thoracotomy tube within the pleural cavity.

U.S. Pat. No. 4,710,171, issued Dec. 1, 1987 to Rosenberg, concerns a needle depth setting sheath assembly and needle stop. The biological needle depth setting arrangement comprises a locking assembly tightenable about the needle barrel. A sheath with graduations therein permits the needle depth to be adjusted accurately. An elastomer bushing is compressible about the needle barrel, and the bushing is prevented from dislocation through a screw-hub arrangement.

Two U.S. patents, U.S. Pat. No. 6,203,524 issued Mar. 20, 2001 and U.S. Pat. No. 6,447,477 issued Sep. 10, 2002 to Burney, illustrate a surgical and pharmaceutical site access guide for biopsy and microtherapy which includes an introducer cannula defining a lumen sized to receive a diagnostic or therapeutic item therethrough and a lateral opening in communication with the lumen adjacent the first end of the cannula. The invention also includes a solid tip having an anatomically distal end secured to the first end of the cannula and a proximal end configured to pierce tissue. A ramp is disposed within the cannula at an end of the lateral opening adjacent the first end of the cannula. The cannula of the introducer preferably includes a plurality of depth markings located in spaced relation along the cannula. These markings are preferably arranged in groups of five to give a ready visual indication of the depth of insertion of the cannula.

U.S. Pat. No. 6,726,684, issued Apr. 27, 2004 to Woloszko, provides methods and apparatus for selectively applying electrical energy to a target location within a patient's body, particularly including tissue in the spine. In a method of the invention high frequency (RF) electrical energy is applied to one or more active electrodes on an electrosurgical probe in the presence of an electrically conductive fluid to remove, contract or otherwise modify the structure of tissue targeted for treatment. In one aspect, a dura mater and spinal cord are insulated from the electrical energy by an insulator positioned on a non-active side of the probe. In another aspect, a plasma is aggressively formed in the electrically conductive fluid by delivering a conductive fluid to a distal end portion of the probe and aspirating the fluid from a location proximal of the return electrode. In another aspect, a distal end of an electrosurgical probe having at least one electrode on a biased, curved, bent, or steerable shaft is guided or steered to a target site within an intervertebral disc having a disc defect for treatment of tissue to be treated at the target site by the selective application of electrical energy thereto. The shaft proximal end portion of electrosurgical probe includes a plurality of depth markings (shown as 903a-f in FIG. 51A). In other embodiments, other numbers and arrangements of depth markings may be included on shaft. For example, in certain embodiments, depth markings may be present along the entire length of shield, or a single depth marking may be present at shaft proximal end portion. Depth markings serve to indicate to the surgeon the depth of penetration of shaft into a patient's tissue, organ, or body, during a surgical procedure. Depth markings may be formed directly in or on shield, and may comprise the same material as shield. Alternatively, depth markings may be formed from a material other than that of shield. For example, depth markings may be formed from materials which have a different color and/or a different level of radiopacity, as compared with material of shield. For example, depth markings may comprise a metal, such as tungsten, gold, or platinum oxide (black), having a level of radiopacity different from that of shield. Such depth markings may be visualized by the surgeon during a procedure performed under fluoroscopy. In one embodiment, the length of introducer needle and shaft are selected to limit the range of shaft distal end beyond the distal tip of introducer needle. FIG. 51B shows a probe, wherein the shaft includes a mechanical stop.

While there are prior art surgical devices with depth markings and stops of various types, what is needed is a surgical device and method having a depth marked cutting instrument with a stop used in conjunction with an ultrasound guided measurement and implementation of the cutting instrument for multiple applications.

### BRIEF SUMMARY OF THE INVENTION

An object of the present invention is to provide a surgical device and method having a depth marked surgical cutting instrument with a stop used in conjunction with an ultrasound guided measurement and implementation of the cutting instrument for multiple applications.

In brief, a surgical cutting instrument which has an ultrasonic reflective coating and gradations thereon is used in association with an adjustable stop ring on the surgical cutting instrument for internal surgical procedures guided by ultrasonic scanning. The surgical cutting instrument is inserted into the introducer and the stop ring stops the surgical cutting instrument at a desired protruding distance of the cutting instrument into the area of the surgical procedure.

An advantage of the present invention is that is provides precise internal surgical procedures mechanically limited by the stop ring against the introducer tube and guided by ultrasonic scanning.

### BRIEF DESCRIPTION OF THE SEVERAL VIEWS OF THE DRAWINGS

These and other details of my invention will be described in connection with the accompanying drawings, which are furnished only by way of illustration and not in limitation of the invention, and in which drawings:

FIG. 1 is a side elevational view of the surgical cutting instrument, introducer tube and trocar used for the ultrasound guided percutaneous common extensor tendon release debridement of the present invention showing how the cutting instrument is inserted in the introducer tube for use replacing the trocar;

FIG. 2 is a partial schematic elevational view of the structure of a patient's elbow showing the introducer tube of FIG. 1 adjacent to the tendon and the surgical cutting instrument extending out of the introducer tube into the area of the surgical procedure;

FIG. 3 is a partial schematic plan view of the structure of a patient's elbow showing the tendon-bone interface being incised starting at the dorsal end of the tendon and proceeding to the ventral side of the tendon, using a fanning style movement of the introducer tube with the surgical cutting instrument extending out the distal end a distance equal to the distance of the ring stop from the base line of FIG. 1;

FIG. 4 is a side elevational view of the surgical cutting instrument, introducer tube and trocar used for the ultrasound guided percutaneous plantar fascia release of the present invention showing how the cutting instrument is inserted in the introducer tube for use replacing the trocar;

FIG. 4A is a top plan view of the surgical cutting instrument of FIG. 4;

FIG. 5 is a partial schematic elevational view of the structure of a patient's heel showing the insertion point of the introducer tube;

FIG. 6 is a partial schematic plan view of the structure of a patient's heel showing the insertion of the introducer tube with the surgical instrument of FIG. 4 extending over the surgical procedure area of the plantar fascia.

### DETAILED DESCRIPTION OF THE INVENTION

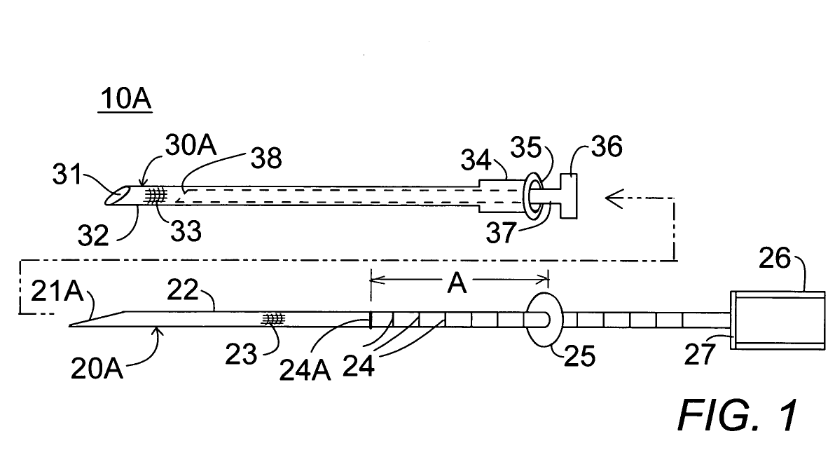

In FIGS. 1-6, a ring stop device and method for a surgical cutting instrument 20A and 20B is used with an introducer tube 30A and 30B in ultrasound guided surgical procedures.

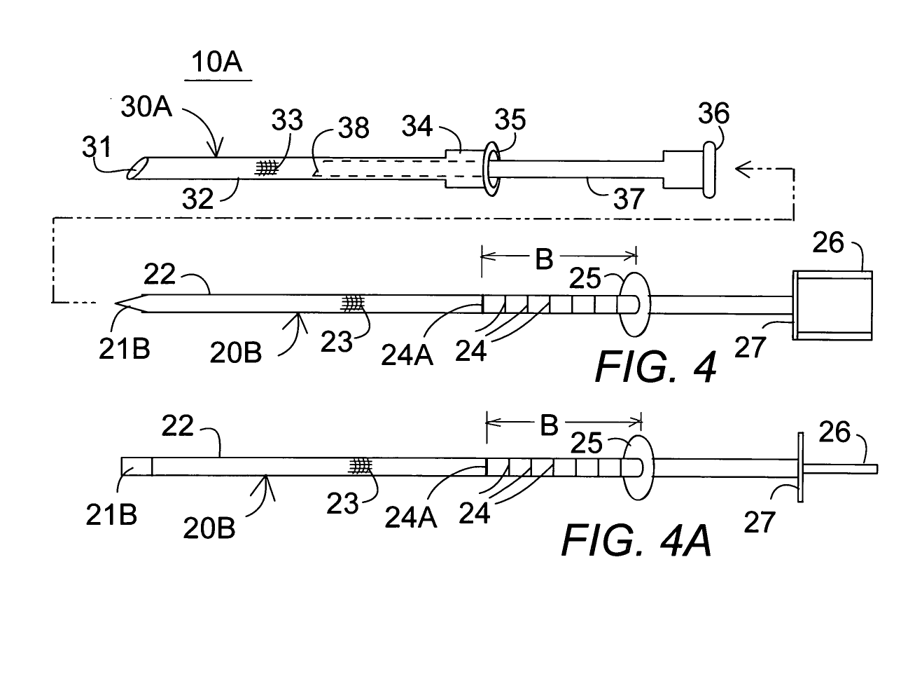

In FIGS. 1 and 4, the introducer tube 30A and 30B comprises a hollow elongated tube with a ring opening 35 and needle receiving pocket 34 at a proximal end and a slanted opening 31 at a distal end, and having an ultrasound reflective coating 33 at least on a distal end portion thereof so that the location of the introducer tube adjacent to an area to receive a surgical procedure within a patient is determined by an ultrasound scan, as shown in FIGS. 2 and 6.

A trocar 37 with a grasping tab 36 at a proximal end and an angled point 38 at a distal end is removably inserted into the introducer tube 30A and 30B to insert the introducer tube into a body of a patient, the trocar being removed after inserting the introducer tube to admit the surgical cutting instrument 20A and 20B into the introducer tube 30A and 30B, as indicated by the arrow in FIGS. 1 and 4..

In FIGS. 1, 4 and 4A, the surgical cutting instrument 20A and 20B comprises an elongated element 22 having a cutting point 21A and 21B for surgical procedures at a distal end for insertion in the introducer tube 30A and 30B with the cutting point extending beyond the introducer tube a sufficient distance to perform a surgical procedure on the area to receive the surgical procedure, as shown in FIGS. 2, 3, and 6. An ultrasound reflective coating 23 coats at least a distal end portion of the elongated element for ultrasound visibility inside a body of a patient to guide the surgical cutting instrument in the surgical procedure.

A calibrated portion 24 along a portion of the length of the elongated element adjacent to a proximal end commences with a base line 24A at a point from the distal end equal to the length of the introducer tube. A stop ring 25 slidably attached to the elongated element in the calibrated portion 24 is set at a desired distance from the base line 24A equal to a desired protruding length of the surgical cutting instrument beyond the distal end of the introducer tube, as shown in FIGS. 2 and 6, to enable the surgical procedure using the cutting point 21A and 21B. The stop ring 25 limits the degree of insertion of the surgical cutting instrument into the introducer tube so that the protruding distal end of the surgical cutting instrument is limited to the length of the area A and B, as shown in FIGS. 2 and 6, to receive the surgical procedure, so that the surgical procedure is guided by an ultrasound scan of the area to receive the surgical procedure inside the patient.

In FIGS. 1-3, the surgical procedure comprises an ultrasound guided percutaneous common extensor tendon 44 release debridement in the elbow region 40 and the stop ring 25 is set at a distance A from the base line 24A equal to the distance A, as shown in FIG. 2, from a proximal part of the tendon origin to a distal aspect of the tendon origin as measured using an ultrasound scan. The introducer tube is 6 cm in length and the base line is located 6 cm from the distal end of the surgical cutting tool.

In FIGS. 4-6, the surgical procedure comprises an ultrasound guided percutaneous plantar fascia 54 release in the heel region 50 and the stop ring 25 is set at a distance B from the base line 24A equal to 30% to 50% of the measured width C of the planter fascia 54 in a transverse plane taken from a point about 2 cm from a fascia origin on a calcaneous 51 as measured and marked on a corresponding area on the skin of the patient at the medial heel as measured using an ultrasound scan. The introducer tube is 8 cm in length and the base line is located 8 cm from the distal end of the surgical cutting tool.

In use, a ring stop method uses a surgical cutting instrument 20A and 20B with an introducer tube 30A and 30B for ultrasound guided surgical procedures.

A first step comprises inserting an introducer tube 30A and 30B into a patient adjacent to an area to receive a surgical procedure within the patient, the introducer tube comprising a hollow elongated tube 32 having an ultrasound reflective coating 33 at least on a distal end portion thereof so that the location of the introducer tube inside the body of the patient is determined by an ultrasound scan.

The first step further comprises inserting a trocar removably in the introducer tube to insert the introducer tube into a body of a patient, the trocar removed after inserting the introducer tube to admit a surgical cutting instrument in the introducer tube.

A second step comprises setting a ring stop 25 on the surgical cutting instrument 20A and 20B into an insertion limiting position on the surgical cutting instrument. The surgical cutting instrument comprises an elongated element 22 having a cutting point 21 A and 21B for surgical procedures at a distal end for insertion in the introducer tube 30A and 30B with the cutting point extending beyond the introducer tube a sufficient distance A and B to perform a surgical procedure on the area to receive the surgical procedure. An ultrasound reflective coating 23 at least on a distal end portion of the elongated element enables ultrasound visibility inside a body of a patient to guide the surgical cutting instrument in the surgical procedure.

A calibrated portion 24 along a portion of the length of the elongated element adjacent to a proximal end commences with a base line 24A at a point from the proximal end equal to the length of the introducer tube. The stop ring 25 is slidably attached to the elongated element in the calibrated portion for setting the stop ring at a desired distance A or B, in FIGS. 1 and 4, from the base line 24A equal to a desired protruding length A and B, in FIGS. 2 and 6, of the surgical cutting instrument 20A and 20B beyond the distal end of the introducer tube to enable the surgical procedure using the cutting point 21A and 21B, the stop ring 25 limiting the degree of insertion of the surgical cutting instrument into the introducer tube so that the protruding distal end of the surgical cutting instrument is limited to the area to receive the surgical procedure.

A third step comprising inserting the surgical cutting instrument 20A and 20B into the introducer tube 30A and 30B to perform the surgical procedure on the area of the patient to receive the surgical procedure inside the patient, the surgical procedure guided by an ultrasound scan of the area inside the patient to receive the surgical procedure.

In FIGS. 1-3, the surgical procedure comprises an ultrasound guided percutaneous common extensor tendon release debridement and the second step comprises setting the stop ring 25 at a distance A from the base line 24A, as shown in FIG. 1, equal to the distance A, as shown in FIG. 2, from a proximal part of the tendon 44 origin to a distal aspect of the tendon origin as measured by the ultrasound scan.

Initially, the procedure involves sterilely doing a lateral elbow/epicondylar field block on the affected elbow. Once local anesthesia is obtained to the affected elbow and is sterility prepped and draped in the usual fashion (fenestrated drape works well). Then using a sterilely covered musculoskeletal ultrasound probe with sterile ultrasound gel, the common extensor tendon 44 is visualized in the longitudinal and transverse plane with elbow bent at about 90° with the arm at the patient's side. At this point the distance A from the proximal part of the tendon origin to the distal aspect of the tendon origin is measured, as shown in FIG. 2 and this distance A set on the cutting instrument 20A with the stop ring 25, as shown in FIG. 1.

Once the cutting instrument stop ring 25 is set, the introducer 30A is placed into the proximal part of the tendon origin, the proximal end of the lateral epicondyle 42, as shown in FIG. 2. The trocar 37 is removed and the cutting instrument 20A is inserted.

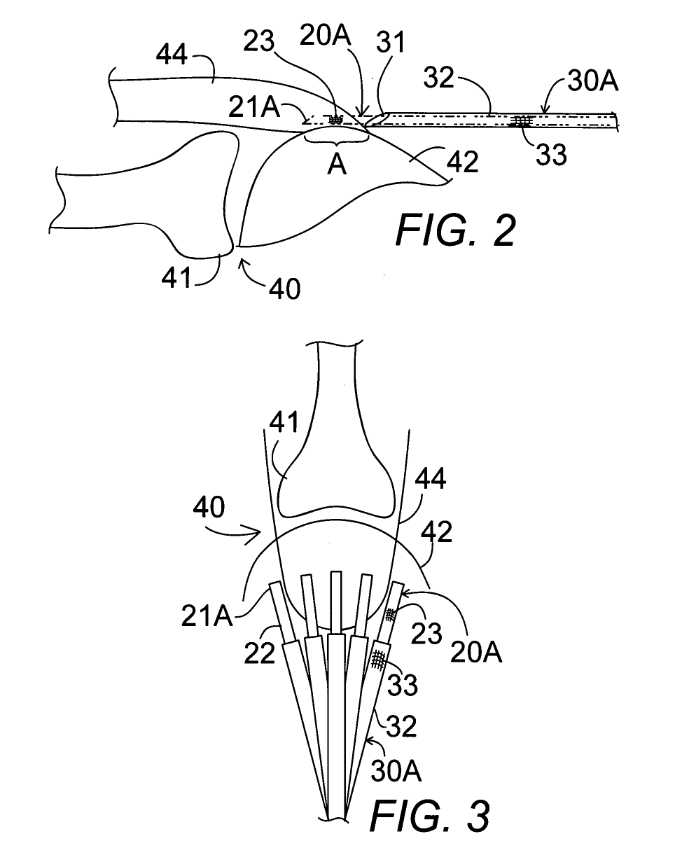

Once the cutting instrument 20A is inserted, the tendon-bone interface is incised starting at the dorsal end of the tendon and proceeding to the ventral side of the tendon, using a fanning style movement of the introducer, as shown in FIG. 3. The cutting instrument is passed into the introducer 20A up to the stop ring 25 with each pass basically debriding the teneosseous junction completely.

Once debridement release is complete, if desired, the cutting instrument 20A is removed and the introducer 30A is left in the tendon. Using approximately 3 cc of previously prepared PRP in a syringe, the syringe is attached to the introducer and injected under ultrasound visualization making sure the PRP infiltrates the tendon (longitudinal visualization is good).

The trocar is replaced and the introducer is removed and a sterile pressure dressing is applied.

For post procedure care, lock up wrist splint at all times except bathing×1 week, then at h.s. for 2 weeks after follow up with doctor, may discontinue after these two weeks. Strengthening, sprayed stretch and cold laser can be added after the 1st week, if desired.

In FIGS. 4-6, the surgical procedure comprises an ultrasound guided percutaneous plantar fascia 54 release and the second step comprises setting the stop ring 25 at a distance B from the base line 24A, as shown in FIG. 4, equal to 30% to 50% of the measured width B of the planter fascia 54, as shown in FIG. 6, in a transverse plane taken from a point about 2 cm from a fascia origin on the calcaneous 51 as measured and marked on a corresponding area on the skin of the patient at the medial heel as measured using an ultrasound scan.

Initially, the procedure involves sterilely doing a posterior tibial nerve block with subsequent local field block medial heel of the affected foot where the trocar is to be inserted. Once local anesthesia is obtained the affected foot/ankle is sterilized, prepped, and draped in the usual fashion.

Using a sterilized covered musculoskeletal ultrasound probe with sterile ultrasound gel the plantar fascia 54 is viewed in the longitudinal and transverse planes on the plantar surface of the heel. The area about 2 cm from the fascia origin on the calcaneous 51 is measured and marked to the corresponding area on the skin (medial heel). At this mark, the planter fascia 54 is visualized in the transverse plane and the width of the fascia is measured using the ultrasound again.

Once the transverse measurement B is determined 30% to 50% of the measured width is taken and going from the distal mark on the cutting device, the ring stop 25 is then moved the number of centimeters indicated on the cutting instrument.

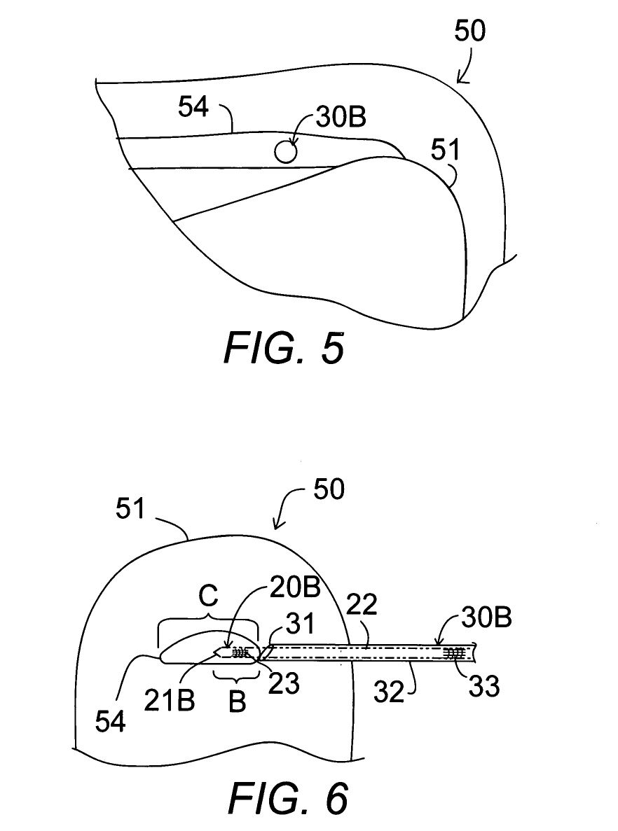

Under ultrasound guidance (longitudinal and then transverse view) the introducer 30B is then inserted perpendicular to the medial heel at the previously determined mark and advanced to the medial edge of the fascia, as shown in FIG. 5.

Once introducer placement is confirmed in the proper location by ultrasound, the trocar is removed and the cutting instrument 20B is inserted to the previously determined spot, as shown in FIG. 6. Again the cutting instrument should be visualized by ultrasound especially in the transverse plain.

The cutting instrument 20B should be removed and the resulting incision in the fascia should be visualized to make sure it is appropriate and acceptable.

(Optional) If PRP is to be introduced after plantar fascial incision, then after the cutting instrument is removed, a previously prepared syringe of about 3 cc of PRP is attached and injected with the introducer still at or slightly moved into the fascia at the site of the incision. Ultrasound guidance should be used (longitudinal) to the PRP permeate the fascia.

The trocar 37 is reinserted then the instrument is removed, and then a sterile pressure dressing is applied.

For post procedural care:

a. Plantar fascia night splint and crunches×3 days, then at h.s.×weeks

b. Ambulation as tolerated after the 3rd day

c. May add cold laser 7× and intrinsic muscle strengthening and PT if desired to help the healing process

It is understood that the preceding description is given merely by way of illustration and not in limitation of the invention and that various modifications may be made thereto without departing from the spirit of the invention as claimed.

What is claimed is:

1. A ring stop device for a surgical cutting instrument used with an introducer tube in ultrasound guided surgical procedures, the device comprising:
an introducer tube comprising a hollow elongated tube having an ultrasound reflective coating at least on a distal end portion thereof so that the location of the introducer tube adjacent to an area to receive a surgical procedure within a patient is determined by an ultrasound scan;
a trocar removably inserted in the introducer tube to insert the introducer tube into a body of a patient, the trocar removed after inserting the introducer tube to admit a surgical cutting instrument in the introducer tube;
a surgical cutting instrument comprising an elongated element having a cutting point for surgical procedures at a distal end for insertion in the introducer tube with the cutting point extending beyond the introducer tube a sufficient distance to perform a surgical procedure on the area to receive the surgical procedure, an ultrasound reflective coating at least on a distal end portion of the elongated element for ultrasound visibility inside a body of a patient to guide the surgical cutting instrument in the surgical procedure, a calibrated portion along a portion of the length of the elongated element adjacent to a proximal end commencing with a base line at a point from the distal end equal to the length of the introducer tube, a stop ring slidably attached to the elongated element in the calibrated portion for setting the stop ring at a desired distance from the base line equal to a desired protruding length of the surgical cutting instrument beyond the distal end of the introducer tube to enable the surgical procedure using the cutting point, the stop ring limiting the degree of insertion of the surgical cutting instrument into the introducer tube so that the protruding distal end of the surgical cutting instrument is limited to the area to receive the surgical procedure, so that the surgical procedure is guided by an ultrasound scan of the area to receive the surgical procedure inside the patient.

2. The device of claim 1 wherein the surgical procedure comprises an ultrasound guided percutaneous common extensor tendon release debridement and the stop ring is set at a distance from the base line equal to the distance from a proximal part of the tendon origin to a distal aspect of the tendon origin as measured using an ultrasound scan.

3. The device of claim 2 wherein the introducer tube is 6 cm in length and the base line is located 6 cm from the distal end of the surgical cutting tool.

4. The device of claim 1 wherein the surgical procedure comprises an ultrasound guided percutaneous plantar fascia release and the stop ring is set at a distance from the base line equal to 30% to 50% of the measured width of the planter fascia in a transverse plane taken from a point about 2 cm from a fascia origin on a calcaneous as measured and marked on a corresponding area on the skin of the patient at the medial heel as measured using an ultrasound scan.

5. The device of claim 4 wherein the introducer tube is 8 cm in length and the base line is located 8 cm from the distal end of the surgical cutting tool.

6. A ring stop method for a surgical cutting instrument used with an introducer tube in ultrasound guided surgical procedures, the method comprising:
a first step comprising inserting an introducer tube into a patient adjacent to an area to receive a surgical procedure within the patient, the introducer tube comprising a hollow elongated tube having an ultrasound reflective coating at least on a distal end portion thereof so that the location of the introducer tube inside a body of the patient is determined by an ultrasound scan;
the first step further comprising inserting a trocar removably in the introducer tube to insert the introducer tube into a body of a patient, the trocar removed after inserting the introducer tube to admit a surgical cutting instrument in the introducer tube;
a second step comprising setting a ring stop on a surgical cutting instrument into an insertion limiting position on the surgical cutting instrument, the surgical cutting instrument comprising an elongated element having a cutting point for surgical procedures at a distal end for insertion in the introducer tube with the cutting point extending beyond the introducer tube a sufficient distance to perform a surgical procedure on the area to receive the surgical procedure, an ultrasound reflective coating at least on a distal end portion of the elongated element for ultrasound visibility inside a body of a patient to guide the surgical cutting instrument in the surgical procedure, a calibrated portion along a portion of the length of the elongated element adjacent to a proximal end commencing with a base line at a point from the distal end equal to the length of the introducer tube, the stop ring slidably attached to the elongated element in the calibrated portion for setting the stop ring at a desired distance from the base line equal to a desired protruding length of the surgical cutting instrument beyond the distal end of the introducer tube to enable the surgical procedure using the cutting point, the stop ring limiting the degree of insertion of the surgical cutting instrument into the introducer tube so that the protruding distal end of the surgical cutting instrument is limited to the area to receive the surgical procedure; and
a third step comprising inserting the surgical cutting instrument into the introducer tube to perform the surgical procedure on the area of the patient to receive the surgical procedure inside the patient, the surgical procedure guided by an ultrasound scan of the area inside the patient to receive the surgical procedure.

7. The method of claim 6 wherein the surgical procedure comprises an ultrasound guided percutaneous common extensor tendon release debridement and the second step comprises setting the stop ring at a distance from the base line equal to a distance from a proximal part of the tendon origin to a distal aspect of the tendon origin as measured by the ultrasound scan.

8. The method of claim 4 wherein the surgical procedure comprises an ultrasound guided percutaneous plantar fascia release and the second step comprises setting the stop ring at a distance from the base line equal to 30% to 50% of the measured width of the planter fascia in a transverse plane taken from a point about 2 cm from a fascia origin on a calcaneous as measured and marked on a corresponding area on the skin of the patient at the medial heel as measured using an ultrasound scan.
